(19) United States
(12) Patent Application Publication
(10) Pub. No.: US 2010/0125238 A1
(43) Pub. Date: May 20, 2010

(54) Iontophoretic Therapeutic Agent Delivery System

(75) Inventors: Whye-Kei Lye, San Jose, CA (US); Kareen Looi, San Jose, CA (US)

Correspondence Address: PATENT LAW OFFICE OF DAVID G. BECK, P. O. BOX 1146, MILL VALLEY, CA, 94942, US

(73) Assignee: THERAWIRE, INC., San Jose, CA (US)

(21) Appl. No.: 12/614,399

(22) Filed: Nov. 7, 2009

Related U.S. Application Data
(60) Provisional application No. 61/199,354, filed on Nov. 14, 2008.
(60) Provisional application No. 61/205,676, filed on Jan. 22, 2009.

(51) Int. Cl. A61N 1/30

(52) U.S. Cl. 604/21

(57) ABSTRACT

A system for delivering one or more therapeutic agents contained on or within a delivery segment through a passageway, e.g., a blood vessel, for treatment of a localized region of the passageway, or for treatment of region adjacent to the localized region of the passageway, is provided.

### CROSS-REFERENCE TO RELATED APPLICATION

This application claims the benefit of the filing date of U.S. Provisional Patent Application Ser. No. 61/199,354, filed Nov. 14, 2008, and U.S. Provisional Patent Application Ser. No. 61/205,676, filed Jan. 22, 2009, the disclosures of which are incorporated herein by reference for any and all purposes.

### FIELD OF THE INVENTION

The present invention relates generally to drug delivery systems and, in particular, to iontophoretic drug delivery systems.

### BACKGROUND OF THE INVENTION

Peripheral arterial disease (PAD) affects over 8 million Americans, with complications ranging from pain and discomfort in the extremities to more severe conditions such as gangrene which may require amputation of the affected limb or limbs. In 2004, approximately 3.2 million diagnostic and therapeutic interventional peripheral vascular disease procedures were performed in the United States. By 2009, the number of procedures is expected to grow to 4.1 million.

Therapeutic intervention is applied in cases where prescription drugs and lifestyle changes are ineffective, and generally take the form of balloon catheterization followed by elective stenting. This approach, followed by adjunctive mechanical support to prevent abrupt closures from vessel recoil, provides immediate restoration of normal blood flow and vessel patency. Despite these measures, however, restenosis or the re-blockage of the affected vessels may occur, thus requiring additional catheterizations or surgical intervention.

Drug-eluting balloon catheters have been introduced as a method to address this problem and help achieve longer term vessel patency. While this is a nascent field, recent clinical studies have shown that the delivery of paclitaxel from the surface of a drug-coated balloon can significantly reduce restenosis in coronary as well as peripheral arteries. As physical contact is the mode of drug delivery in drug-eluting balloon catheters, a particular problem with this approach is that a portion of the drug will typically be lost from the surface of the balloon as it is threaded across complex and tortuous lesions prior to deployment. As a result of losing some of the therapeutic agent prior to reaching the intended delivery site, a sub-optimal or poorly defined drug payload will be administered upon balloon deployment.

To overcome some of the problems associated with placing the drug or other therapeutic agent on the outside of the balloon catheter, another approach uses a permeable or semi-permeable balloon catheter. For example, U.S. Pat. No. 5,286,254 discloses the use of either a single or double balloon catheter in which the intended drug is placed in solution, that solution then being used to inflate the balloon catheter once it is in position. The pressure of the drug solution within the balloon causes the drug solution to be transported across the walls of the balloon and into direct contact with the vessel wall. In one disclosed embodiment, the system uses iontophoresis in combination with pressure to drive the drug solution through the walls of the balloon catheter.

Although there are a variety of techniques and systems that provide localized delivery of a drug using an arterial catheter, these techniques and systems tend to have limited efficacy due to the delivery mode, and limited applicability due to the size of the catheter. Accordingly, what is needed is a drug delivery system that allows accurate delivery of the intended drug to the desired site for a wide range of vessel sizes, and further allows drug delivery to be localized within a region of the desired site. The present invention provides such a drug delivery system.

### SUMMARY OF THE INVENTION

The present invention provides a system for delivering one or more therapeutic agents contained on or within a delivery segment through a passageway, e.g., a blood vessel, for treatment of a localized region of the passageway, or for treatment of region adjacent to the localized region of the passageway.

In at least one embodiment of the invention, an iontophoretic therapeutic agent delivery system for localized delivery of a therapeutic agent to internal body tissue is provided, the system comprised of (i) a flexible guide wire comprised of a body segment and at least one delivery segment; (ii) a first polymer coating covering the body segment of the guide wire, the first polymer coating being fabricated from an electrically non-conductive material; (iii) a second polymer coating covering the at least one delivery segment, wherein the therapeutic agent is infused into the second polymer coating, or at least a portion thereof; (iv) means for conducting an electrical signal from a proximal end of the guide wire to the therapeutic agent delivery segment; and (v) means for applying the electrical signal to the conducting means, wherein application of the electrical signal causes migration of the therapeutic agent from the delivery segment to the internal body tissue. The body segment and the therapeutic agent delivery segment preferably have diameters of 0.1 inches or less, and more preferably 0.035 inches or less. The flexible guide wire may include a lumen. The means for applying the electrical signal to the conducting means may be comprised of a programmable power supply. The flexible guide wire may comprise the means for conducting the electrical signal to the therapeutic agent delivery segment. The flexible guide wire may be comprised of a material selected from the group consisting of stainless steel, nitinol, cobalt chromium alloys, or an alloy containing one or more of iron, nickel, platinum, rhodium, palladium, magnesium, aluminum, gold, silver, vanadium, tungsten, chromium, cobalt, titanium, ruthenium, iridium or osmium. The first polymer coating may be comprised of a material selected from the group consisting of polytetrafluoroethylene, polyvinyl chloride, polyethylene, polyimide, parylene, polyester or nylon. The second polymer may be comprised of a material selected from the group consisting of polyethylene glycol, poly(acrylic acid), poly(2-hydroxy ethyl methacrylate), poly(N-vinyl pyrrolidone), poly(methyl methacrylate), poly(vinyl alcohol), polyacrylamide, poly(ethylene-co-vinyl acetate), poly(ethylene glycol), poly(methacrylic acid), polylactides, polyglycolides, poly(lactide-co-glycolides), polyanhydrides, polysiloxanes, polyphosphazenes, poly(ethylene imines), poly(alkylene sulphides), poly(propiolactones), cellulose acetates, poly(vinyl methyl ketones), polystyrenes, polyorthoesters, chitosan gels, hydrogels or any combination thereof.

The system may further comprise at least one individually addressable electrode within the at least one therapeutic agent delivery segment, wherein the at least one conductor corresponds to the at least one electrode and is configured to conduct electrical signals from the proximal end of the flexible guide wire to the at least one electrode. The system may further comprise a layer of electrically insulating material interposed between each of the at least one electrodes and the flexible guide wire. The system may further comprise a layer of electrically insulating material interposed between each of the at least one conductors and the flexible guide wire. The system may further comprise an indicator located on the proximal end of the flexible guide wire, the indicator having a known alignment with the at least one electrodes. The therapeutic agent may be infused into one or more regions of the second polymer coating, the regions aligned with the at least one electrode.

The system may further comprise an adjustable sleeve configured to be mounted on the patient undergoing treatment with the iontophoretic therapeutic agent delivery system, wherein the adjustable sleeve is comprised of a plurality of electrodes configured to be coupled to the electrical signal applying means, and wherein the electrical signal applying means applies power to each of the plurality of electrodes in a predetermined order.

The system may further comprise at least one therapeutic agent delivery segment marker, for example a radio-opaque marker locatable by fluoroscopy.

The system may further comprise a balloon catheter proximal to the at least one therapeutic agent delivery segment, and means for inflating and deflating the balloon catheter.

The system may further comprise means, for example an expandable wire cage, for centering the at least one therapeutic agent delivery segment within a body passageway.

In at least one embodiment of the invention, an iontophoretic therapeutic agent delivery system for localized delivery of a therapeutic agent to internal body tissue is provided, the system comprised of (i) a flexible guide wire comprised of a body segment and at least one delivery segment; (ii) a first polymer coating covering the body segment and the at least one delivery segment of the guide wire, the first polymer coating being fabricated from an electrically non-conductive material; (iii) a second polymer infused with the therapeutic agent, the second polymer contained within a lumen within the at least one therapeutic agent delivery segment; (iv) a plurality of apertures coupling the lumen and the second polymer contained within the lumen to an exterior surface of the at least one therapeutic agent delivery segment; (v) means for conducting an electrical signal from a proximal end of the guide wire to the therapeutic agent delivery segment; and (vi) means for applying the electrical signal to the conducting means, wherein application of the electrical signal causes migration of the therapeutic agent from the second polymer within the lumen to the internal body tissue.

A further understanding of the nature and advantages of the present invention may be realized by reference to the remaining portions of the specification and the drawings.

### BRIEF DESCRIPTION OF THE DRAWINGS

FIG. 1 schematically illustrates the therapeutic delivery system of the present invention;

FIG. 2 illustrates a cross-sectional view of the therapeutic delivery segment;

FIG. 3 illustrates a cross-sectional view of a therapeutic delivery segment similar to that shown in FIG. 2, except without the guidewire lumen;

FIG. 4 provides an exterior view of a portion of a therapeutic delivery guide wire system in which the therapeutic agent is constrained to a specific region of the therapeutic delivery segment;

FIG. 5 provides an exterior view of a portion of a therapeutic delivery guide wire system in which the electrode within the wire guide comprises only a portion of the wire guide core;

FIG. 6 provides a cross-sectional view of the delivery segment of the system shown in FIG. 5 utilizing a solid core guide wire;

FIG. 7 provides a cross-sectional view of the delivery segment of the system shown in FIG. 5 utilizing a hollow core guide wire;

FIG. 8 provides a cross-sectional view of the delivery segment of a system using both a localized electrode as illustrated in FIGS. 6 and 7, and a therapeutic agent comprising only a region of the delivery segment as illustrated in FIG. 4;

FIG. 9 provides a cross-sectional view of a delivery segment using multiple, separately addressable electrodes;

FIG. 10 provides a cross-sectional view of a delivery segment with multiple, separately addressable electrodes as shown in FIG. 9, and multiple regions of therapeutic agent corresponding to the separate electrodes;

FIG. 11 provides a perspective view of an adjustable sleeve member that includes multiple, independent electrodes;

FIG. 12 illustrates an iontophoretic drug delivery system that includes multiple, individually addressable delivery segments;

FIG. 13 provides a cross-sectional view of the drug delivery system shown in FIG. 12 along plane A-A;

FIG. 14 provides a cross-sectional view of the drug delivery system shown in FIG. 12 along plane B-B;

FIG. 15 is an illustration of the therapeutic delivery system shown in FIG. 1 with markers positioned immediately before and after the drug delivery segment;

FIG. 16 is an illustration of the therapeutic delivery system shown in FIG. 1 with an inflated balloon catheter positioned immediately before the drug delivery segment;

FIG. 17 illustrates the use of expandable wire cages to center the drug delivery segment within the vessel or passageway, the expandable wire cages being shown in the collapsed state;

FIG. 18 illustrates the expandable wire cages of FIG. 17 in the expanded state;

FIG. 19 illustrates an iontophoretic drug delivery system in which the drug infused polymer is located within a lumen of the guide wire core;

FIG. 20 provides a cross-sectional view of the drug delivery system shown in FIG. 19 along plane A-A;

FIG. 21 illustrates the therapeutic delivery system shown in FIG. 2 used with a primary guide wire in an OTW configuration; and

FIG. 22 illustrates the therapeutic delivery system shown in FIG. 2 used with a primary guide wire in a RX configuration.

### DESCRIPTION OF THE SPECIFIC EMBODIMENTS

In the following text, the terms “drug” and “therapeutic agent” may be used interchangeably and may refer to a small molecule drug, a protein, metal ions, non-metallic anions, RNA, DNA, or some combination thereof. The term “drug” and “therapeutic agent” may also refer to nanoscale constructs such as nanoparticles, dentritic molecules and/or micellular bodies that are used to encapsulate a small molecule drug, a protein, metal ions, non-metallic anions, RNA, DNA, or some combination thereof. Examples of small molecule drugs that may be delivered include, but are not limited to, tissue plasminogen activator (tPA), urokinase, paclitaxel, sirolimus, everolimus, zotarolimus, tacrolimus, vincristine, prednisone, dexamethasone, heparin, hirudin, dexamethaxone, atorvastatin, ETC-216 (apoA-1 Milano), and/or clopidogrel. The functional classes of therapeutic agents that may be delivered include, but are not limited to, anti-restenotic agents, chemotherapy agents, anti-inflammatory agents, vasodilators, thrombolytics, and/or HMG-CoA reductase inhibitors (statins). It should be understood that identical element symbols used on multiple figures refer to the same component, or components of equal functionality. Additionally, the accompanying figures are only meant to illustrate, not limit, the scope of the invention and should not be considered to be to scale.

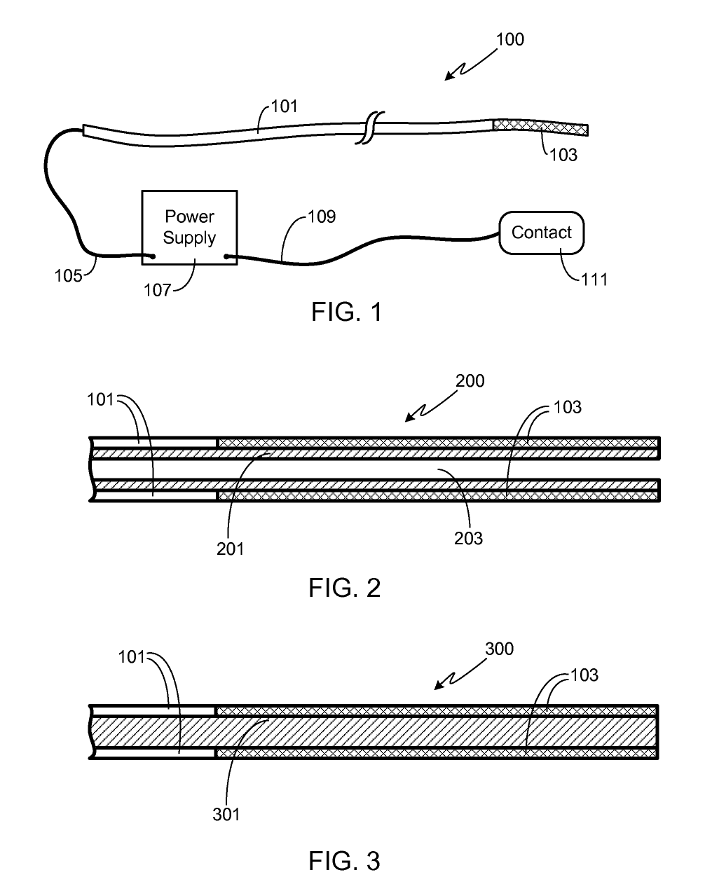

In general, and as illustrated in FIG. 1, an iontophoretic therapeutic agent delivery system 100 fabricated in accordance with the invention includes a primary body segment 101 and at least one therapeutic agent delivery segment 103 that is generally located at the distal end of the guide wire. Delivery system 100 is designed to allow one or more therapeutic agents contained on or within delivery segment 103 to be delivered through a passageway, e.g., a blood vessel, for treatment of a localized region of the passageway, or for treatment of region adjacent to the localized region of the passageway. Due to the size of delivery system 100, preferably less than 0.1 inches and more preferably in the range of 0.014 inches to 0.035 inches (i.e., between 300 microns and 900 microns), a therapeutic agent delivery system in accordance with the present invention can be introduced into relatively small passages, for example blood vessels that are too small to allow passage of a balloon catheter. Accordingly, longer and tighter lesions in the peripheral vascular and neurovascular systems may be treated by the invention. Additionally, it will be appreciated that a system in accordance with the invention can also be used to deliver therapeutic agents to other lumens within the body. While it is expected that in a typical application of the invention, the therapeutic agents contained on or within the delivery segment 103 will be directed towards and into the local vessel wall by electrokinetic forces as described further below, it will be appreciated that any therapeutic agents released into the blood stream may also be used to treat tissues and organs that are reached by circulation distal to the point of release. For example, in this mode of operation, the drug delivery system of the invention may be utilized to deliver chemotherapeutic agents directly into tumors immediately adjacent or distal to the point of drug release. Furthermore, the iontophoretic mechanism may enhance tissue uptake of these agents further increasing therapeutic efficiency.

Release of the drug or other therapeutic agent contained on or within delivery segment 103 is triggered by application of an electrical stimulus. Preferably, the necessary electric field is generated by coupling one electrode 105 of a suitable power supply 107 (e.g., a programmable power supply) to the conductive core of guide wire 100, and coupling a second electrode 109 to a contact 111 that is in contact with the patient. Contact 111 may consist of an electrode attached to the patient's skin, for example using an adhesive patch, or an implantable, transdermal electrode.

FIGS. 2 and 3 provide cross-sectional views of two different designs for a guide wire therapeutic delivery system as described herein, each of these views including the therapeutic delivery segment 103 and a small portion of the primary body segment 101. Within guide wire 200 is a conductive guide wire core 201 that includes a guide wire lumen 203. Within guide wire 300 is a solid-core, conductive guide wire core 301. The conductive guide wire core (e.g., core 201, core 301) of the therapeutic delivery system is electrically connected to electrode 105 of power supply 107 as previously noted. Exemplary materials suitable for use as the conductive guide wire core include, but are not limited to, 316L stainless steel, nitinol, cobalt chromium alloys such as MP35N or L605, or any suitable alloy containing one or more of iron, nickel, platinum, rhodium, palladium, magnesium, aluminum, gold, silver, vanadium, tungsten, chromium, cobalt, titanium, ruthenium, iridium or osmium. Therapeutic delivery segment 103 is comprised of a polymer impregnated with the desired therapeutic agent or agents, the polymer being ion conductive and capable of maintaining the therapeutic agent(s) in a charged form. Suitable polymers include, but are not limited to, polyethylene glycol (PEG), poly(acrylic acid) PAA, poly(2-hydroxy ethyl methacrylate), poly(N-vinyl pyrrolidone), poly(methyl methacrylate), poly(vinyl alcohol), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polysiloxanes, polyphosphazenes, poly(ethylene imines), poly(alkylene sulphides), poly(propiolactones), cellulose acetates, poly(vinyl methyl ketones), polystyrenes, polyorthoesters, chitosan gels, hydrogels or any combination thereof. The non-therapeutic agent containing portion of the guide wire delivery system, i.e., guide wire body segment 101 is comprised of an electrically insulating material that overcoats the guide wire core. Suitable electrically insulating coatings include, but are not limited to, a polymer such as polytetrafluoroethylene (PTFE), polyvinyl chloride (PVC), polyethylene, polyimide, parylene, polyester or nylon.

After the therapeutic delivery segment(s) is positioned at the intended delivery site, an electrical stimulus is applied to the guide wire, causing the release and delivery of the therapeutic agent. Typically, the electrical stimulus also enhances penetration of the therapeutic agent into the tissue that is proximate to the delivery segment. As contact between the delivery segment 103 and the area to be treated is not required to deliver the therapeutic agent, it is possible to minimize, if not altogether eliminate, procedurally related trauma such as that which often accompanies the use of a balloon drug delivery catheter. As a result, the risk of restenosis is decreased and the ability to treat the same location multiple times is improved, a clear benefit for a number of medical conditions that require multiple doses of one or more therapeutic agents.

The electrical stimulus applied via power supply 107 to the therapeutic segment 103 may be in the form of a constant direct current, a square wave, triangular wave, rectangular wave, sinusoidal wave, saw-toothed wave, rectified sinusoidal wave, etc. Almost any waveform may be used, subject to the condition that it effect therapeutic delivery into the vessel wall without causing pain or injury to the patient. Other operational parameters that may be varied include voltage, current and frequency. These parameters may be physician controlled or a processor within power supply 107 may be preprogrammed with the desired operational parameters. The selected values for these operational parameters depend upon the specifics of the therapeutic delivery segment (e.g., segment diameter and length), passageway or vessel size, dimensions of the area to be treated, impedance of the system/patient, ability of the patient to withstand the generated electric field (e.g., pre-existing heart or other medical conditions that may affect the operational parameters), dose requirements for the selected therapeutic agent (e.g., single/multiple doses, dose frequency and duration), polarity of the therapeutic agent, etc. Preferably the selected current density is in the range of 1 fA/cm² to 1 A/cm², more preferably in the range of 1 μA/cm² to 100 mA/cm², and still more preferably in the range of 10 μA/cm² to 10 mA/cm². Preferably the selected frequency is in the range of 0 Hz to 1 GHz, more preferably in the range of 10 Hz to 1 MHz, and still more preferably in the range of 20 kHz to 100 kHz. In general, and making reasonable assumptions regarding lesion length and vessel diameter, the inventors have found that for most applications a current output capability of 15 mA is sufficient for power supply 107.

The polarity of the bias applied to the therapeutic delivery segment depends on the selected therapeutic. If the therapeutic agent has a neutral charge, as is the case with paclitaxel, it may be necessary to encapsulate the therapeutic agent in a charged micelle. The charge of such micelles depends upon the molecules comprising the micelle and the surrounding media. For instance, if the micelle is composed of SDS (sodium dodecyl sulfate) and is present in an aqueous solution, it will carry a negative charge. In this instance, the guidewire will be coupled to the negative potential of power supply 107. Under these conditions, the paclitaxel molecules will be transported from the drug delivery segment 103, which has a negative potential, into the vessel wall that is held at a positive potential.

The application of an electrical stimulus to effect drug delivery necessitates the consideration that hydrolysis of water may occur in-vivo. Water hydrolyzes at approximately 1.7 V and generates H⁺ and O²⁻ ions that may alter the local pH in the region. Some therapeutic agents, such as the limus family of macro-cyclic lactones (sirolimus etc), are susceptible to cleavage under acidic conditions, with the resulting product exhibiting significantly lower efficacy than the parent compound. To address this, a pH buffer solution may be formulated into the polymer to mitigate the effect of water hydrolysis on therapeutic efficacy. Alternatively, a polymer with an inherent pH buffering capability may also be used as the drug repository.

One of the benefits of the present therapeutic delivery system, as opposed to a contact delivery system, is that the amount of therapeutic agent delivered from the guide wire is easily controlled by adjusting the magnitude and duration of the current applied by supply 107 during the procedure. More specifically, the total dose, D, will be proportional to the integral:

D∝∫I(t)dt

where I(t) is the current as a function of time. It will be appreciated that within a single percutaneous intervention, multiple iontophoretic dosings may be applied and that the total dose within the intervention will be proportional to the sum of those dosings.

Directional Therapeutic Delivery

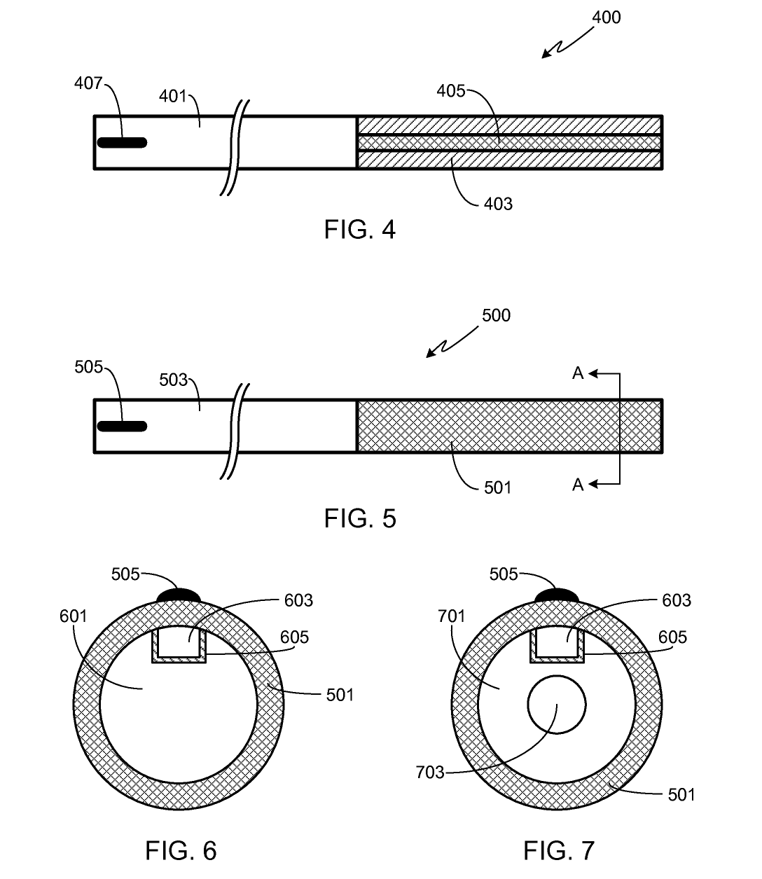

In the previously described embodiment, upon application of the electrical stimulus, the therapeutic agent contained on or within delivery segment 103 is directed radially outwards from the segment. It will be appreciated that for some applications it may be desirable to preferentially direct the therapeutic agent in one or more selected directions. One method of accomplishing this goal is to apply the therapeutic agent to only a portion of the delivery segment. For example, FIG. 4 is an external view of a portion of guide wire therapeutic delivery system 400 showing the therapeutic delivery segment 403 and a small portion of the primary body segment 401 adjacent to delivery segment 403 and a small portion of the proximal end portion of the primary body segment 401, segment 401 containing no therapeutic agents. Therapeutic delivery segment 403 includes a region 405 that includes the selected therapeutic agent while the remaining portion of segment 403 contains no therapeutic agent. Located at the proximal end of guide wire 400 is an indent, slot, colored marker, bump or other indicator 407 that is aligned with region 405, thus allowing the physician or operator to properly locate the region containing the therapeutic agent adjacent to the area to be treated. It will be appreciated that this approach may be used with either a lumen containing guide wire as shown in FIG. 2, or a solid core guide wire as shown in FIG. 3.

Another approach to delivering the therapeutic agent to a selected location proximate to the delivery segment is to localize the electrode within the guide wire. Localizing the electrode within the guide wire core causes localization of the field generated between this electrode and the oppositely charged vessel wall. As a result, the therapeutic agent is primarily delivered at a site adjacent to the wire guide core electrode, tapering off as the distance from this electrode increases. FIG. 5 provides an exterior view of a therapeutic agent guide wire delivery system 500 in accordance with this embodiment of the invention. As shown, delivery system 500 is comprised of the delivery segment 501, the primary body segment 503 (only a portion of which is shown) and an indicator 505 such as an indent, slot, colored marker, bump or other indicator that is aligned with the localized electrode. FIGS. 6 and 7 are cross-sectional views taken along plane A-A through the distal end portion of segment 501, FIG. 6 based on a solid core guide wire 601 and FIG. 7 based on a hollow core guide wire 701. Each of these views show the localized electrode 603. It will be understood that the localized electrode runs the full length of the guide wire, thus allowing it to be coupled to at the proximal end of the guide wire. Assuming that the guide wire core is conductive, a layer 605 of electrically insulating material separates electrode 603 from the core. It will be understood that if the core is not electrically conductive, layer 605 of electrically insulating material is not required. In both of these views indicator 505 is visible. It will be appreciated, however, that if indicator 505 is formed by an indent, mark, slot, or other indicator that does not extend away from the body of the guide wire, it would not be visible in either FIG. 6 or FIG. 7. Note that the guide wire core lumen is indicated by reference 703 in FIG. 7. It should also be appreciated that the localized electrode, i.e., electrode 603, may utilize a different shape or comprise a different proportion of the core than illustrated.

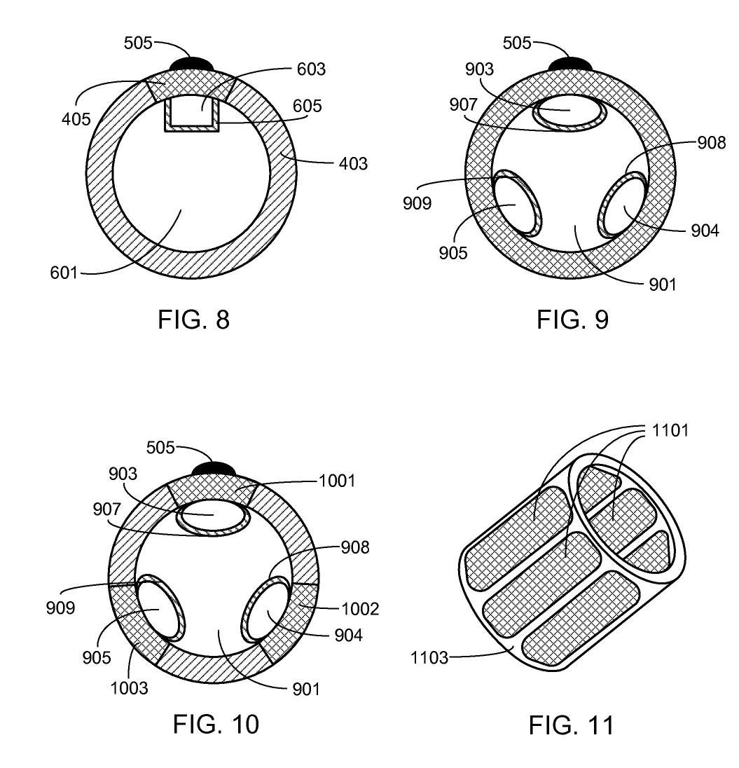

FIG. 8 is a cross-sectional view of a therapeutic agent delivery segment that combines the features of the embodiments of FIGS. 4 and 5. It should be understood that while FIG. 8 illustrates the use of a solid core guide wire, a hollow guide wire as illustrated in FIG. 7 could also be used. As shown, adjacent to electrode 603 is region 405 containing the selected therapeutic agent. The remaining portion 403 of the delivery segment contains no therapeutic agent.

In another embodiment, the guide wire contains multiple electrodes, each individually addressable at the proximal end of the assembly. For example, in the cross-sectional view of a delivery segment shown in FIG. 9, guide wire core 901 includes three separate electrodes 903-905, each electrode running the length between the therapeutic agent delivery region and the proximal end where the electrodes are coupled to the power supply (e.g., supply 107). It will be appreciated that a fewer, or a greater, number of electrodes may be employed depending upon the needs of the patient and the drug therapy prescribed by the physician. Although electrodes 903-905 have a different shape than electrode 603, it will be appreciated that either electrode shape, or yet another electrode shape, may be used. Assuming a conductive guide wire core, electrodes 903-905 are electrically isolated using insulators 907-909, respectively, as shown. Alternately, if a non-conductive guide wire core is used, electrical insulating layers 907-909 are not required. Although a single indicator 505 is shown in this figure, it will be appreciated that multiple indicators may be used, each corresponding to one of the electrodes. Additionally and as previously noted, any of a variety of markers may be used for indicator 505, each providing the physician/technician with the ability to orient the drug delivery regions with respect to the regions to be treated. It will be appreciated that while this embodiment is illustrated in FIG. 9 with a solid core guide wire, this embodiment may be used equally well with a hollow core guide wire such as that shown in FIGS. 2 and 7.

While the use of individual, separately addressable electrodes such as that described above and illustrated in FIG. 9 allows drug delivery to be directional rather than circumferential around the periphery of the delivery segment, further directionality may be achieved by confining the therapeutic agent to selected regions of the delivery segment. Preferably, the regions containing the therapeutic agent are adjacent to the electrode as illustrated in FIG. 10. As shown, adjacent to electrodes 903-905 are regions 1001-1003, each containing a therapeutic agent and separated from an adjacent drug-containing region. Regions 1001-1003 may contain the same therapeutic agent, or different therapeutic agents.

Multi-Electrode Delivery System

In the present invention, iontophoresis causes the migration of the therapeutic agent to the adjacent, and oppositely charged, vessel wall. Since one of the electrodes comprising the electrical stimulus circuit is attached to, or implanted within, the patient, the electric field generated around the periphery of the drug delivery segment may be non-uniform. Accordingly, the inventors have found that the use of multiple, sequentially energized electrodes may be used to improve field uniformity, and thus drug delivery uniformity.

It will be appreciated that there are countless ways in which multiple electrodes may be positioned such that they approximately surround the drug delivery segment of the guide wire based, iontophoretic delivery system of the invention. For example, multiple adhesive patches, each of which includes an electrode, may be attached to the patient approximately surrounding the region to be treated. FIG. 11 provides a perspective view of an alternate approach in which multiple and independent electrodes 1101 are coupled to an adjustable sleeve member 1103. Sleeve member 1103 may be fabricated from a stretchable material (e.g., neoprene). Alternately, sleeve member 1103 may be configured to include an adjustable buckle or other means of adjustment. In use, sleeve 1103 is positioned around the area to be treated, for example the patient's leg, and then adjusted to ensure contact between the electrodes 1101 and the patient's skin. During treatment, the power supply coupled to the electrodes is configured to apply power to each electrode individually, preferably in a sequential pattern. It will be appreciated that in addition to providing a means for achieving improved field uniformity, the use of multiple electrodes as shown may also be used to provide a non-uniform drug treatment, for example by applying power to some of the electrodes for longer periods of time, thus increasing the dosage in the corresponding regions.

Differential Lateral Therapeutic Delivery

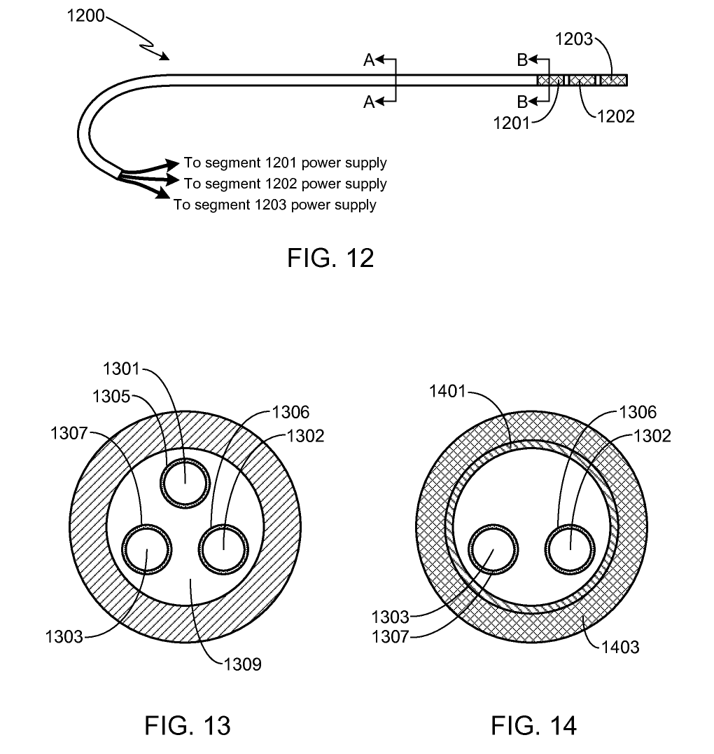

In the embodiments described relative to FIGS. 4-10, directional delivery of the therapeutic agent is achieved through the use of individually addressable electrodes and/or non-uniform placement of the therapeutic agent in the delivery segment. FIG. 12 illustrates an iontophoretic drug delivery system that includes multiple, individually addressable delivery segments 1201-1203, thus providing differential therapeutic agent delivery along the length of the guide wire. It will be appreciated that the system may use fewer, or greater, numbers of delivery segments; that the spacing between segments may or may not be uniform; that each delivery segment may use an electrode that covers the entire circumference of the guide wire core for that segment (e.g., as shown in FIGS. 2 and 3) or a localized electrode (e.g., as shown in FIGS. 6 and 7) or multiple electrodes (e.g., as shown in FIG. 9); and that each delivery segment may distribute the therapeutic agents within the delivery segment uniformly or non-uniformly (e.g., as shown in FIGS. 8 and 10).

In general, the guide wire core of system 1200 includes multiple conductive elements that couple the electrode or electrodes within each drug delivery segment to electrical connectors at the proximal end of the guide wire, thus allowing the electrodes of the delivery segments to be coupled to a suitable power supply (e.g., power supply 107). FIG. 13 provides a cross-sectional view of therapeutic guide wire delivery system 1200 taken along plane A-A. As shown, three conductive elements 1301-1303 with outer electrical insulators 1305-1307 run through guide wire core 1309. It will be appreciated that different conductor configurations may be used, for example, different conductive element shapes, sizes or number (e.g., if one or more of the delivery segments includes multiple electrodes or if multiple delivery segments are coupled to the same conductive element). Additionally, insulators 1305-1307 are only required if the guide wire core is comprised of an electrically conductive material. Although not shown, guide wire core 1309 may include a lumen as previously described relative to FIG. 2. Assuming that the guide wire core is fabricated from an electrically conductive material, this portion of the delivery system is coated with an electrically insulating material 1311, for example a polymer such as PTFE, PVC, polyethylene, polyimide, parylene, polyester or nylon.

FIG. 14 provides a cross-sectional view of delivery segment 1201 taken along plane B-B. In this view, only conductive elements 1302 and 1303 are shown as element 1301 is coupled to delivery segment electrode 1401. Note that the connection between element 1301 and electrode 1401 is not shown in this view. Surrounding electrode 1401 is a layer 1403 of the therapeutic agent.

Therapeutic Delivery Segment Location Markers

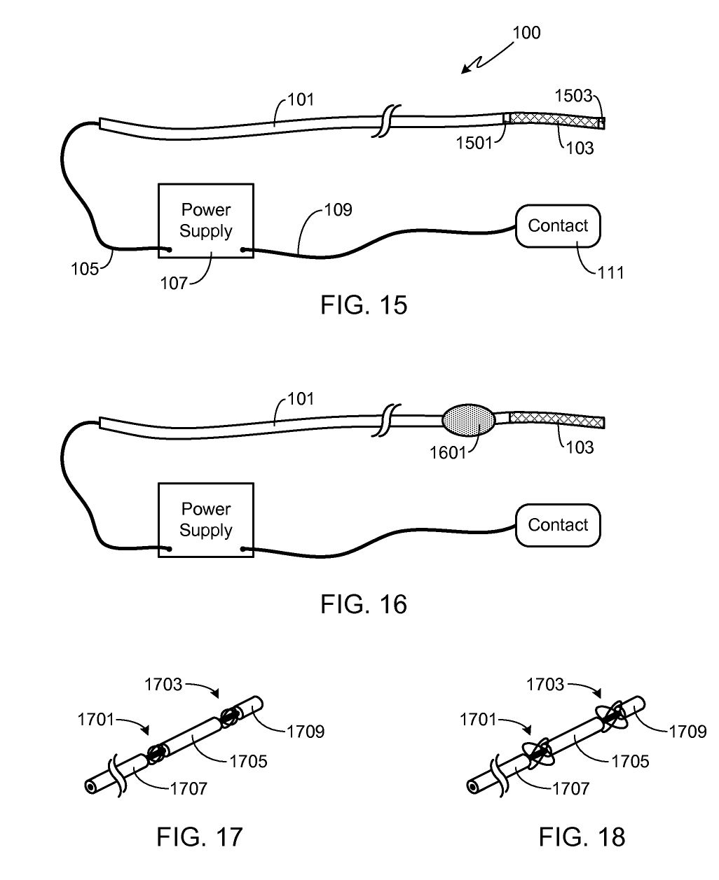

Any of the embodiments disclosed herein may utilize markers to aid in positioning the delivery segment(s) at the location to be treated. For example, FIG. 15 is an illustration of therapeutic delivery system 100 with markers 1501 and 1503 positioned immediately before and after, respectively, drug delivery segment 103, thereby delineating the proximal and distal ends of segment 103. It will be appreciated that a single marker may be used, for example located before, after, or within the delivery segment.

Preferably markers 1501 and 1503 are radio-opaque markers that can be located using fluoroscopy. Accordingly, markers 1501 and 1503 may be comprised of gold, platinum or similar material known in the field. The markers can be placed over, under, or within the material comprising either the body segment (e.g., segment 101) or the drug delivery segment (e.g., segment 103). Regardless of the location of the markers, preferably they do not alter the cross-sectional profile of the device. In at least one embodiment, the radio-opaque markers are used as an aid in determining the orientation of the delivery segment(s) under fluoroscopic guidance.

In at least one embodiment, the guide wire core is comprised of at least two different materials that are distinguishable by fluoroscopy. One of the materials underlies body segment 101 while a second of the materials, preferably the more radio-opaque material, underlies the drug delivery segment 103.

Therapeutic Delivery System with Balloon Catheter

Any of the embodiments disclosed herein may utilize a balloon catheter positioned before the drug delivery segment. For example, FIG. 16 is an illustration of therapeutic delivery system 100 with an inflated balloon catheter 1601 positioned before drug delivery segment 103. A proximal balloon catheter such as that illustrated in FIG. 16 may be used to occlude or reduce blood flow through the treated vessel or stenosis during the iontophoretic procedure. As necessary, the balloon catheter can be inflated and deflated to occlude and reperfuse the vessel during the procedure to prevent ischemia.

Self-Centering Therapeutic Delivery System

Any of the embodiments disclosed herein may include one or more structures, preferably located on either side of the drug delivery segment, that center the drug delivery segment within the vessel when activated. Although balloon catheters may be used for this purpose, they are inappropriate for many applications as they occlude the vessel when expanded. Accordingly in at least one embodiment of the invention, located on either side of the drug delivery segment is an expandable wire cage. An exemplary wire cage is shown in FIGS. 17 and 18. In the illustrated example, the wires comprising cages 1701 and 1703 are shown in the collapsed state in FIG. 17, and in the expanded state in FIG. 18. Preferably in the collapsed state the cages have a cross-section the same as, or similar to, drug delivery segment 1705 and body segment 1707. Although not required, in the exemplary structure of FIGS. 17 and 18 there is a small body segment 1709 distal to drug delivery segment 1705.

In at least one embodiment, the wires comprising cages 1701 and 1703 are fabricated from a nickel-titanium (Nitinol) alloy or a similar material that contracts when subjected to a low level current. Thus in an exemplary structure, the ends of the cages are constrained to the guide wire, causing the cages to collapse as shown in FIG. 17 when subjected to an electrical stimulus. Accordingly during treatment, the electrical stimulus is removed from the cages, causing them to expand and center the drug delivery segment. Then, after treatment, electrical stimulus is applied causing the cages to collapse, thereby allowing the therapeutic drug delivery system to be withdrawn. It will be appreciated that self-centering cages may utilize a variety of different designs, activation schemes and materials. For example, the present invention may employ self-centering cages where the cages expand upon application of electrical stimulus, and collapse upon removal of the electrical stimulus.

Therapeutic Agent Captured within the Guide Wire Core

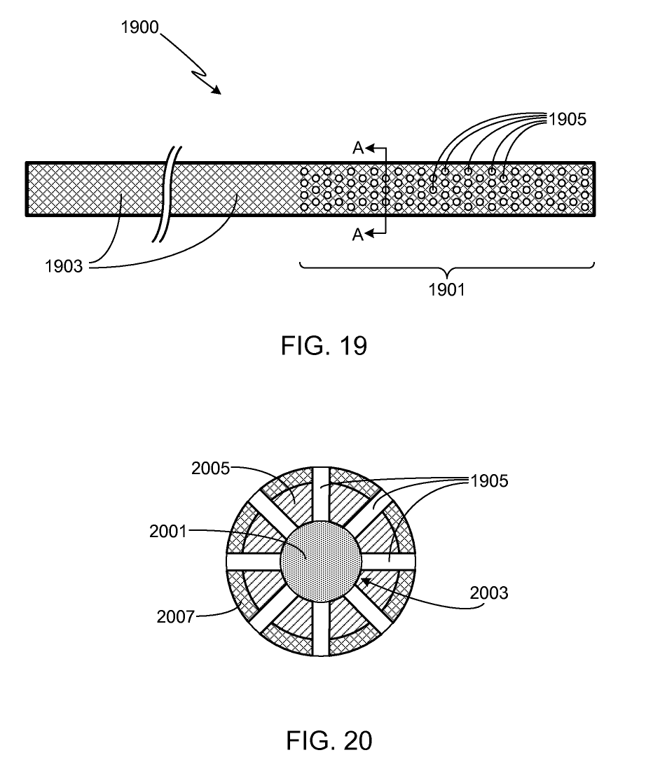

In the embodiments described above, the guide wire core of the drug delivery segment is coated with a polymer coating that is infused with the desired therapeutic agent or agents. FIGS. 19 and 20 illustrate an alternative approach, FIG. 19 providing an exterior view of a therapeutic delivery system 1900 that is comprised of a drug delivery segment 1901 and a body segment 1903, and FIG. 20 providing a cross-sectional view of drug delivery segment 1901 along plane A-A. As shown, the drug infused polymer 2001 is located within a lumen 2003 of the guide wire core 2005. The exteriors of both the body segment and the drug delivery segment are coated with an electrically insulating polymer 2007, for example a polymer such as PTFE, PVC, polyethylene, polyimide, parylene, polyester or nylon. A plurality of holes or slots 1905 are formed in the drug delivery segment 1901, for example using laser machining or electrical discharge machining (EDM). As in the previous embodiments, application of an electrical stimulus causes the therapeutic agent, in this case located within lumen 2003, to migrate out of the device and into the vessel wall.

In an alternative to the above approach, the polymer and therapeutic agent are physically separate, but disposed within guide wire lumen 2003 in such a fashion that the physical expansion of the polymer will force the drug out of the device. In this embodiment, the polymer may be a chitosan gel, which are known to expand/contract upon application of an electric current. As before, therapeutic delivery into the vessel wall may be affected by diffusion or enhanced by the iontophoretic or electrophoretic mechanisms.

Iontophoretic Catheter System

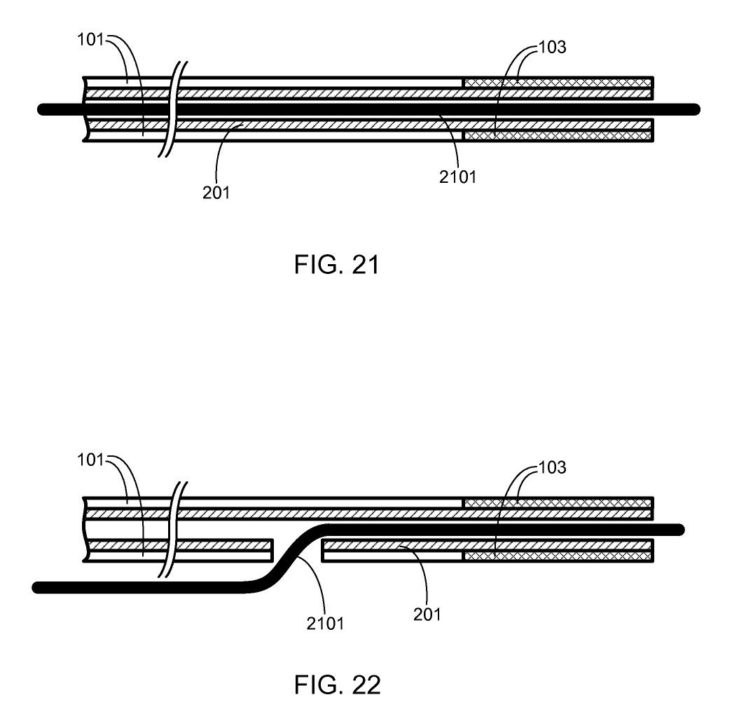

While it is envisioned that the iontophoretic therapeutic system of the invention may be configured as a guide wire and be utilized by physicians as a primary guide wire for clinical procedures, in an alternate embodiment the iontophoretic therapeutic system of the invention may be configured as an over-the-wire (OTW) or a rapid-exchange (RX) catheter system. In these embodiments, the central lumen (e.g., lumen 203 of FIG. 2, lumen 703 of FIG. 7, etc.) serves as a conduit for the primary guide wire, allowing the physician to use the iontophoretic therapeutic system in conjunction with the primary guide wire of their choice. FIGS. 21 and 22 illustrate OTW and RX systems, respectively, based on system 200 and a primary guide wire 2100. It will be appreciated that any of the previously disclosed embodiments may be configured to include a lumen for use with a primary guide wire, e.g., guide wire 2100.

As will be understood by those familiar with the art, the present invention may be embodied in other specific forms without departing from the spirit or essential characteristics thereof. Accordingly, the disclosures and descriptions herein are intended to be illustrative, but not limiting, of the scope of the invention which is set forth in the following claims.

1. An iontophoretic therapeutic agent delivery system for localized delivery of a therapeutic agent to internal body tissue, comprising:
a flexible guide wire, said flexible guide wire comprised of a body segment and at least one therapeutic agent delivery segment;
a first polymer coating covering said body segment of said flexible guide wire, wherein said first polymer coating is electrically non-conductive;
a second polymer coating covering said at least one therapeutic agent delivery segment of said flexible guide wire, wherein the therapeutic agent is infused into said second polymer coating;
means for conducting an electrical signal from a proximal end of said flexible guide wire to said at least one therapeutic agent delivery segment; and
means for applying said electrical signal to said conducting means, wherein application of said electrical signal to said conducting means causes migration of the therapeutic agent from said at least one therapeutic agent delivery segment to said internal body tissue.

2. The iontophoretic therapeutic agent delivery system of claim 1, wherein said body segment has a diameter of 0.1 inches or less, and wherein said at least one therapeutic agent delivery segment has a diameter of 0.1 inches or less.

3. The iontophoretic therapeutic agent delivery system of claim 1, wherein said body segment has a diameter of 0.035 inches or less, and wherein said at least one therapeutic agent delivery segment has a diameter of 0.035 inches or less.

4. The iontophoretic therapeutic agent delivery system of claim 1, said flexible guide wire further comprising a guide wire lumen.

5. The iontophoretic therapeutic agent delivery system of claim 1, wherein said means for applying said electrical signal to said conducting means is comprised of a programmable power supply.

6. The iontophoretic therapeutic agent delivery system of claim 1, wherein said flexible guide wire comprises said means for conducting said electrical signal.

7. The iontophoretic therapeutic agent delivery system of claim 1, wherein said flexible guide wire is comprised of a material selected from the group consisting of stainless steel, nitinol, cobalt chromium alloys, or an alloy containing one or more of iron, nickel, platinum, rhodium, palladium, magnesium, aluminum, gold, silver, vanadium, tungsten, chromium, cobalt, titanium, ruthenium, iridium or osmium.

8. The iontophoretic therapeutic agent delivery system of claim 1, wherein said first polymer coating is comprised of a material selected from the group consisting of polytetrafluoroethylene, polyvinyl chloride, polyethylene, polyimide, parylene, polyester or nylon.

9. The iontophoretic therapeutic agent delivery system of claim 1, wherein said second polymer coating is comprised of a material selected from the group consisting of polyethylene glycol, poly(acrylic acid), poly(2-hydroxy ethyl methacrylate), poly(N-vinyl pyrrolidone), poly(methyl methacrylate), poly(vinyl alcohol), polyacrylamide, poly(ethylene-co-vinyl acetate), poly(ethylene glycol), poly(methacrylic acid), polylactides, polyglycolides, poly(lactide-co-glycolides), polyanhydrides, polysiloxanes, polyphosphazenes, poly(ethylene imines), poly(alkylene sulphides), poly(propiolactones), cellulose acetates, poly(vinyl methyl ketones), polystyrenes, polyorthoesters, chitosan gels, hydrogels or any combination thereof.

10. The iontophoretic therapeutic agent delivery system of claim 1, wherein the therapeutic agent is infused into a first portion of said second polymer coating, and wherein a second portion of said second polymer coating is not infused with said therapeutic agent.

11. The iontophoretic therapeutic agent delivery system of claim 10, further comprising an indicator located on said proximal end of said flexible guide wire and aligned with said first portion of said second polymer coating.

12. The iontophoretic therapeutic agent delivery system of claim 1, further comprising an electrode within said flexible guide wire and within said at least one therapeutic agent delivery segment, wherein said conducting means further comprises a conductor contained within said flexible guide wire and said body segment, and wherein said conductor conducts said electrical signal from said proximal end of said flexible guide wire to said electrode.

13. The iontophoretic therapeutic agent delivery system of claim 12, further comprising a layer of electrically insulating material separating said electrode from said flexible guide wire.

14. The iontophoretic therapeutic agent delivery system of claim 12, further comprising a layer of electrically insulating material separating said conductor from said flexible guide wire.

15. The iontophoretic therapeutic agent delivery system of claim 12, further comprising an indicator located on said proximal end of said flexible guide wire and aligned with said electrode.

16. The iontophoretic therapeutic agent delivery system of claim 12, wherein the therapeutic agent is infused into a first portion of said second polymer coating, wherein a second portion of said second polymer coating is not infused with said therapeutic agent, and wherein said first portion of said second polymer coating is aligned with said electrode.

17. The iontophoretic therapeutic agent delivery system of claim 1, further comprising a plurality of electrodes within said flexible guide wire and within said at least one therapeutic agent delivery segment, wherein said conducting means further comprises a plurality of conductors corresponding to said plurality of electrodes and contained within said flexible guide wire and said body segment, and wherein each of said plurality of electrodes is individually addressable via said plurality of conductors.

18. The iontophoretic therapeutic agent delivery system of claim 17, further comprising a layer of electrically insulating material interposed between each of said plurality of electrodes and said flexible guide wire.

19. The iontophoretic therapeutic agent delivery system of claim 17, further comprising a layer of electrically insulating material interposed between each of said plurality of conductors and said flexible guide wire.

20. The iontophoretic therapeutic agent delivery system of claim 17, further comprising an indicator located on said proximal end of said flexible guide wire, wherein said indicator has a known alignment with said plurality of electrodes.

21. The iontophoretic therapeutic agent delivery system of claim 17, wherein the therapeutic agent is infused into a plurality of regions of said second polymer coating, and wherein said plurality of regions of said second polymer coating are aligned with said plurality of electrodes.

22. The iontophoretic therapeutic agent delivery system of claim 1, further comprising an adjustable sleeve configured to be mounted on a patient undergoing treatment with the iontophoretic therapeutic agent delivery system, wherein said adjustable sleeve is comprised of a plurality of electrodes configured to be coupled to said electrical signal applying means, and wherein said electrical signal applying means applies power to each of said plurality of electrodes in a predetermined order.

23. The iontophoretic therapeutic agent delivery system of claim 1, wherein said at least one therapeutic agent delivery segment is comprised of a plurality of therapeutic agent delivery segments, wherein said conducting means further comprises a plurality of conductors corresponding to said plurality of therapeutic agent delivery segments and contained within said flexible guide wire and said body segment, and wherein each of said plurality of therapeutic agent delivery segments is individually addressable via said plurality of conductors.

24. The iontophoretic therapeutic agent delivery system of claim 23, further comprising a layer of electrically insulating material interposed between each of said plurality of conductors and said flexible guide wire.

25. The iontophoretic therapeutic agent delivery system of claim 1, further comprising at least one therapeutic agent delivery segment marker.

26. The iontophoretic therapeutic agent delivery system of claim 25, wherein said at least one therapeutic agent delivery segment marker is a radio-opaque marker locatable by fluoroscopy.

27. The iontophoretic therapeutic agent delivery system of claim 1, further comprising a balloon catheter proximal to said at least one therapeutic agent delivery segment, and means for inflating and deflating said balloon catheter.

28. The iontophoretic therapeutic agent delivery system of claim 1, further comprising means for centering said at least one therapeutic agent delivery segment within a body passageway.

29. The iontophoretic therapeutic agent delivery system of claim 28, wherein said centering means is comprised of at least one expandable wire cage.

30. The iontophoretic therapeutic agent delivery system of claim 29, wherein said at least one expandable wire cage expands when subjected to an electrical stimulus.

31. An iontophoretic therapeutic agent delivery system for localized delivery of a therapeutic agent to internal body tissue, comprising:
a flexible guide wire, said flexible guide wire comprised of a body segment and at least one therapeutic agent delivery segment;
a first polymer coating covering said body segment and said at least one therapeutic agent delivery segment of said guide wire, wherein said first polymer coating is electrically non-conductive;
a second polymer infused with the therapeutic agent, wherein said second polymer is contained within a lumen within said at least one therapeutic agent delivery segment of said flexible guide wire;
a plurality of apertures coupling said lumen and said second polymer contained within said lumen to an exterior surface of said at least one therapeutic agent delivery segment;
means for conducting an electrical signal from a proximal end of said flexible guide wire to said at least one therapeutic agent delivery segment; and
means for applying said electrical signal to said conducting means, wherein application of said electrical signal to said conducting means causes migration of the therapeutic agent from said second polymer within said lumen of said at least one therapeutic agent delivery segment to said internal body tissue.
